(19) United States
(12) Patent Application Publication
(10) Pub. No.: US 2012/0252715 A1
(43) Pub. Date: Oct. 4, 2012

(54) Dissolvable, Personal Cleansing Compositions

(76) Inventors: Shawn David McConaughy, Cincinnati, OH (US); Edward Dewey Smith, III, Mason, OH (US); Karl Shiqing Wei, Mason, OH (US); Binwu Tao, Beijing (CN)

(21) Appl. No.: 13/439,613

(22) Filed: Apr. 4, 2012

Related U.S. Application Data
(60) Provisional application No. 61/471,631, filed on Apr. 4, 2011.

(51) Int. Cl. A61K 8/81; A61Q 19/10; A61Q 5/02; A61K 8/02

(52) U.S. Cl. 510/438

(57) ABSTRACT

A solid, dissolvable cleansing composition is disclosed. The cleansing composition includes a viscosity of about 1,000,000 to about 50,000,000 Pascal-seconds (PaS). The cleansing composition comprises about 25% or more, by weight of the cleansing composition, of a surfactant with an active content of about 60% or greater and about 5% or more, by weight of the cleansing composition, of a co-surfactant. The cleansing composition further includes about 30% or more, by weight of the cleansing composition, of expandable microspheres.

### CROSS REFERENCE TO RELATED APPLICATIONS

This application claims the benefit of U.S. Provisional Application No. 61/471,631 filed Apr. 4, 2011 which is incorporated herein by reference.

### BACKGROUND

Cleansing the skin and/or hair is an activity that has been done for many years. Over to time, skin and/or hair cleansing have involved the use of liquid cleansing compositions such as liquid soaps and body washes, which tend to be very desirable to consumers due to their cleansing properties, lathering characteristics, mildness, and/or benefit or moisturization delivery. Although widely used and popular, such liquid cleansing compositions tend to require more resources to package, store, transport, and even use. For example, such liquid cleansing compositions are typically sold in bottles that add cost as well as packaging waste. Additionally, such liquid cleansing compositions usually include a substantial amount of water, which increases the weight and size of the compositions, and, thus, the shipping and storage costs thereof.

To reduce some of the resources associated with packaging, storing, transporting, and use of typical liquid cleansing compositions, single dose, dissolvable personal cleansing compositions, such as dissolvable personal cleansing films and dissolvable, porous, solid personal cleansing compositions, have been developed. Unfortunately, such compositions currently available today are not necessarily suited to provide the criteria that are desirable to consumers for liquid cleansing compositions including good cleansing properties, lathering characteristics, benefit delivery, and dissolvability. For example, such dissolvable personal cleansing compositions have formulation limitations and tend to be limited to only particular types of surfactants which have inferior cleansing properties and lathering characteristics of the composition. Additionally, such single dose, personal cleansing compositions available today tend to be difficult to dissolve and costly to manufacture. Thus, there remains a need for a dissolvable, personal cleansing composition that provides the criteria desired by consumers, tends to be easily dissolvable, and/or also tends to be less cost prohibitive to manufacture.

### SUMMARY

In one embodiment, a cleansing composition includes about 15% or more, by weight of the cleansing composition, of a surfactant; about 0.5% or more, by weight of the cleansing composition, of a co-surfactant; and about 5% or more, by weight of the cleansing composition, of expandable microspheres, wherein the cleansing composition comprises a viscosity of about 1,000,000 to about 50,000,000 Pascal-seconds (PaS).

In another embodiment, a solid, dissolvable cleansing composition includes about 25% or more, by weight of the cleansing composition, of a surfactant; about 3% or more, by weight of the cleansing composition, of a co-surfactant; and about 5% or more, by weight of the cleansing composition, of expandable microspheres, wherein the expandable microspheres comprise hollow deformable particles of an expanded copolymer of vinylidene chloride and acrylonitrile; or of vinylidene chloride, acrylonitrile and methacrylate; or a non-expanded copolymer of vinylidene chloride and acrylonitrile; or of vinylidene chloride, acrylonitrile and methacrylate; and wherein the cleansing composition comprises a viscosity of about 1,000,000 to about 10,000,000 Pascal-seconds (PaS).

In another embodiment, a solid, dissolvable cleansing composition includes about 30% or more, by weight of the cleansing composition, of a surfactant, and wherein the surfactant comprises sodium laureth-n sulfate (SLEnS) where n is about 1 to about 3; about 5% or more, by weight of the cleansing composition, of a co-surfactant, wherein the co-surfactant comprises a betaine; and about 6% or more, by weight of the cleansing composition, of expandable microspheres, wherein the expandable microspheres comprise hollow deformable particles of an expanded copolymer of vinylidene chloride and acrylonitrile or of vinylidene chloride, acrylonitrile and methacrylate or a non-expanded copolymer of vinylidene chloride and acrylonitrile or of vinylidene chloride, acrylonitrile and methacrylate, and wherein the cleansing composition comprises a viscosity of about 1,000,000 to about 10,000,000 Pascal-seconds (PaS).

These and other embodiments will be described in more detail below.

### DETAILED DESCRIPTION

While the specification concludes with the claims particularly pointing and distinctly claiming the invention, it is believed that the present invention will be better understood from the following description.

The devices, apparatuses, methods, components, and/or compositions of the present invention can include, consist essentially of, or consist of, the components of the present invention as well as other ingredients described herein. As used herein, “consisting essentially of” means that the devices, apparatuses, methods, components, and/or compositions may include additional ingredients, but only if the additional ingredients do not materially alter the basic and novel characteristics of the claimed devices, apparatuses, methods, components, and/or compositions.

All percentages and ratios used herein are by weight percent of active component in the composition and all measurements made are at 25° C., unless otherwise designated.

All measurements used herein are in metric units unless otherwise specified.

The amounts of components as listed herein is by active level unless otherwise indicated.

The term “cleansing composition” as used herein, refers to compositions intended for topical application to a surface such as skin or hair to, for example, remove dirt, oil, and the like. The personal cleansing compositions disclosed herein can be rinse-off formulations, in which the product is applied topically to the skin or hair via, for example, and then is subsequently rinsed within seconds to minutes from the skin or hair with water. According to example embodiments, the cleansing composition disclosed herein can exhibit a viscosity of about 1,000,000 to about 50,000,000 Pascal-seconds (PaS), alternatively about 1,000,000 to about 20,000,000 PaS, and alternatively about 1,000,000 to about 10,000,000 PaS, and a dissolution rate of about 0.30 mg (surfactant)/s.

As used herein, “dissolvable” means that the cleansing composition meets the hand dissolution value. According to example embodiments, the personal cleansing compositions disclosed herein comprises a hand dissolution value of from about 1 to about 30 strokes, in one embodiment from about 2 to about 25 strokes, in another embodiment from about 3 to about 20 strokes, and in still another embodiment from about 4 to about 15 strokes, as measured by the Hand Dissolution Method.

As used herein, “solid” as used herein means that the cleansing composition disclosed herein can be dry and not flowable i.e. not liquid or semi-liquid prior to use of the cleansing composition.

The term “water-activated,” as used herein, means that the present invention is presented to the consumer in a form to be used with water. It is found that when the cleansing compositions disclosed herein include a surfactant, the compositions produce a lather or are “activated” upon contact with water and further agitation.

### I. Dissolvable Personal Cleansing Composition

One embodiment disclosed herein relates to a solid, dissolvable cleansing composition. The solid, dissolvable cleansing such as the cleansing composition disclosed herein can be used by a consumer in, for example, a shower or bath to cleanse, moisturize, exfoliate, and the like.

The cleansing composition can be a viscoelastic surfactant or soap paste, an elastic surfactant or soap paste, a powder surfactant or soap paste, or any other composition or paste that exhibits suitable lather, viscosity, cleansing, elasticity, and dissolution. In example embodiments, the cleansing composition can include one or more of the following: surfactants, expandable microspheres, electrolytes, and other optional ingredients including preservatives, salts, benefit agents, anti-microbials such as ZPT or zinc pyrithione, exfoliates, actives, fragrances or perfumes, colorants, and the like, all of which will be described in more detail below. For example, in one embodiment, the cleansing composition can be a viscoelastic or elastic solid, dissolvable cleansing composition that can include one or more surfactants and expandable microspheres.

The cleansing composition such as the cleansing composition disclosed herein can also exhibit suitable lathering or cleansing properties, can be a single dose composition such that the cleansing composition is used in its entirety after one shower and/or bath, for example, and can be elastic or viscoelastic such that the cleansing composition can exhibit a viscosity of about 1,000,000 to about 50,000,000 Pascal-seconds (PaS), alternatively about 1,000,000 to about 20,000,000 PaS, and alternatively about 1,000,000 to about 10,000,000 PaS.

According to one embodiment, the cleansing composition disclosed herein can be water activated. For example, the solid, dissolvable cleansing composition can be wetted and agitated to produce lather and provide cleansing, moisturization, and other benefits to the surface such as skin and/or hair the composition is applied to.

A. Surfactants and Co-Surfactants

As described above, in one embodiment, the solid, dissolvable cleansing composition can include a surfactant or a mixture of surfactants such as a surfactant and a co-surfactant suitable for application to a surface that can be used to deliver cleansing properties and also lathering characteristics of the cleansing composition when, for example, the dissolvable, personal cleansing composition is wetted and agitated during use thereby dissolving the expandable microspheres that can structure the surfactant or mixture of surfactants, which will be described in more detail below.

According to an example embodiment, the cleansing composition can include about 10% or greater, alternatively about 20% or greater, and alternatively about 40% or greater, by weight of the cleansing composition, of a surfactant. In another example embodiment, the cleansing composition can include from about 15% to about 99%, alternatively from about 20% to about 90%, and alternatively from about 25% to about 70%, by weight of the cleansing composition, of a surfactant.

The cleansing composition can also include a co-surfactant in combination with the surfactant as described above. For example, in one embodiment, the cleansing composition can include about 0.1% or greater, alternatively about 1% or greater, and alternatively about 10% or greater, by weight of the cleansing composition, of a co-surfactant. According to another embodiment, the cleansing composition can include from about 0.1% to about 20%, alternatively about 1% to about 15%, and alternatively about 2% to about 10%, by weight of the cleansing composition, of a co-surfactant.

In an example embodiment, the surfactant or mixture of surfactant raw materials can include other ingredients such as water that can be a byproduct of processing the surfactant or mixture thereof. According to such an embodiment, the surfactant and/or co-surfactant that can be included in the cleansing composition disclosed herein can have an active content of the actual surfactant or co-surfactant of about 10.0% or greater, alternatively about 30.0% or greater, alternatively about 50.0% or greater, alternatively about 60% or greater, and alternatively about 70.0% or greater.

Suitable surfactants for use in the cleansing composition disclosed herein include anionic, nonionic, cationic, zwitterionic, amphoteric surfactants, soap, or combinations thereof. For example, according to one embodiment, the surfactant can comprise one or more linear anionic surfactants such as ammonium lauryl sulfate, ammonium laureth sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, potassium lauryl sulfate, and combinations thereof.

In one embodiment, the surfactant comprises sodium laureth(n) sulfate, hereinafter SLEnS, where n defines the average moles of ethoxylation. Alternatively, in another embodiment, the surfactant comprises sodium trideceth(n) sulfate, hereinafter STnS, wherein n defines the average moles of ethoxylation. According to example embodiments, n for the SLEnS and/or the STnS can range from greater than 0 to 8, alternatively from about 1 to about 3, alternatively about 2, and alternatively about 1. It should be understood that a material such as SLEnS or STnS can comprise a significant amount of molecules which have no ethoxylate, 1 mole ethoxylate, 2 mole ethoxylate, 3 mole ethoxylate, and so on in a distribution which can be broad, narrow or truncated. For example, SLE1S can comprise a significant amount of molecules which have no ethoxylate, 1 mole ethoxylate, 3 mole ethoxylate, and so on in a distribution which can be broad, narrow or truncated and still comprise SLE1S where the average of the distribution is about 1. Similarly, ST2S can comprise a significant amount of molecules which have no ethoxylate, 1 mole ethoxylate, 2 mole ethoxylate, 3 mole ethoxylate, and so on in a distribution which can be broad, narrow or truncated and still comprise ST2S where the average of the distribution is about 2.

The surfactant can also comprise one or more branched anionic surfactants and monomethyl branched anionic surfactants such as sodium trideceth sulfate, sodium tridecyl sulfate, sodium C12-13 alkyl sulfate, and C12-13 pareth sulfate and sodium C12-13 pareth-n sulfate.

As described above, in other example embodiments, the surfactant can comprise nonionic and cationic surfactants. Nonionic surfactants for use in the viscoelastic cleansing composition include those selected from the group consisting of alkyl glucosides, alkyl polyglucosides, polyhydroxy fatty acid amides, alkoxylated fatty acid esters, sucrose esters, amine oxides, and mixtures thereof. Cationic surfactants for use in the cleansing composition include, but are not to limited to, fatty amines, di-fatty quaternary amines, tri-fatty quaternary amines, imidazolinium quaternary amines, and combinations thereof.

The surfactant can further include an amphoteric surfactant, a zwitterionic surfactant, or mixtures thereof. According to an example embodiment, the amphoteric and zwitterionic surfactant can be combined with other surfactants such that the amphoteric and zwitterionic surfactants can be a co-surfactant. Amphoteric surfactant suitable for use in the surfactant of the viscoelastic cleasinging composition described herein include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition include sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products described in U.S. Pat. No. 2,528,378. In an embodiment, the surfactant included in the personal care composition described herein can comprise an amphoteric surfactant that is selected from the group consisting of sodium lauroamphoacetate, sodium cocoamphoactetate, disodium lauroamphoacetate disodium cocodiamphoacetate, and mixtures thereof.

Zwitterionic surfactants suitable for use in the surfactant of the cleansing composition described herein include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. In one embodiment, the zwitterionic surfactant included in the personal care composition described herein can comprise one or more betaines such as cocoamidopropyl betaine.

In one embodiment, the surfactant comprises sodium cocoyl isethionate.

B. Expandable Microspheres

The cleansing composition can further include expandable microspheres in combination with the surfactant as described above. According to an example embodiment, the expandable microspheres can provide structure to surfactants and/or other ingredients of the solid, dissolvable cleansing composition. For example, the expandable microspheres can define spaces between each of the microspheres or particles thereof. In one embodiment, the spaces defined between the expandable microspheres can house surfactants and/or other ingredients of the solid, dissolvable cleansing composition. As such, the expandable microspheres can provide a reservoir or a vehicle for structuring the surfactants and/or other ingredients by housing such surfactants and/or other ingredients in the spaces defined between the microspheres or particles thereof.

The expandable microspheres can also provide a particular rheology or viscosity for the cleansing composition. For example, the expandable microspheres can provide a viscosity of about 1,000,000 to about 50,000,000 Pascal-seconds (PaS), alternatively about 1,000,000 to about 20,000,000 PaS, and alternatively about 1,000,000 to about 10,000,000 PaS for the solid, dissolvable cleansing composition.

According to an example embodiment, the expandable microspheres can be dissolved by water during use of the solid, dissolvable cleansing composition such that surfactants and/or other ingredients included in the solid, dissolvable cleansing composition can be activated and applied to a surface such as skin and/or hair. For example, a consumer can wet the solid, dissolvable cleansing composition with water. As a result of the wetting, the expandable microspheres can be dissolved and the surfactants and/or other ingredients can be exposed. The exposed surfactants and/or other ingredients can then be activated and applied to a surface such as skin and/or air to lather, cleanse, and deliver or deposit benefit agents or other ingredients included in the solid, dissolvable cleansing composition.

The cleansing composition can include about 2% or greater, alternatively about 3% or greater, alternatively about 4% or greater, and alternatively about 5%, or greater, by weight of the cleansing composition, of expandable microspheres. In another embodiment, the cleansing composition can include from about 2% to about 99%, alternatively from about 3% to about 90%, and alternatively about 4% to about 70%, by weight of the cleansing composition, of expandable microspheres.

In an example embodiment, the expandable microspheres can include other ingredients such as water that can be a byproduct of processing the surfactant or mixture thereof. For example, the expandable microspheres included in the cleansing composition disclosed herein can include actual expandable microspheres particles in combination with water whereby the water can help keep the particles from floating in the air during processing. According to such an embodiment, the expandable microspheres that can be included in the cleansing composition disclosed herein can have an active content of the actual expandable microsphere particles of to about 2.0% or greater, alternatively about 5% or greater; alternatively about 6.0% or greater, alternatively about 8% or greater, alternatively about 10.0% or greater, and alternatively about 15.0% or greater.

According to one embodiment, the expandable microspheres can be hollow deformable particles of an expanded copolymer of vinylidene chloride and acrylonitrile or of vinylidene chloride, acrylonitrile and methacrylate or a non-expanded copolymer of vinylidene chloride and acrylonitrile or of vinylidene chloride, acrylonitrile and methacrylate. In another embodiment, the expandable microspheres can be hollow deformable particles of polymers and/or copolymers of the itaconic, citraconic, maleic and fumaric acids and from vinyl acetate or lactate esters.

Examples of suitable expandable microspheres useful in the cleansing composition disclosed herein include EXPANCEL® available from AkzoNobel including 551 DE 12, 551 DE 20, and 551 DE 50, 551 WU, and 091 We 40 D24.

C. Optional Ingredients

The cleansing composition disclosed herein can further include one or more optional ingredients such as polymers, pluronics, salts, antimicrobial agents, actives, brighteners, silica, moisturizers or benefit agents, perfumes or fragrances, colorants, and the like as described below.

i. Polymers

According to one embodiment, the cleansing composition can include one or more polymers. The one or more polymers can be used to, for example, structure the cleansing composition disclosed herein, to modify rheology of the cleansing composition disclosed herein, to improve lather, skin feel, deposition of benefit agents or antimicrobial agent included in the cleansing composition disclosed herein, and the like. In one embodiment, the one or more polymers can be hydrophobically modified polymers or other suitable structuring polymers, cationic polymers, hydrophilic polymers, deposition polymers, and the like. For example, in one embodiment, the cleansing composition can optionally include from about 0.01% to about 10%, alternatively from about 0.1% to about 8%, and alternatively from about 0.1% to about 5%, by weight of the cleansing composition, of a hydrophobically modified polymer. Suitable hydrophobically modified polymers useful in the cleansing composition disclosed herein can include Acrylates/Vinyl Isodecanoate Crosspolymer (Stabylen 30 from 3V), Acrylates/C10-30 Alkyl Acrylate Crosspolymer (Pemulen TR1 and TR2), Ammonium Acryloyldimethyltaurate/Beheneth-25 Methacrylate Crosspolymer (Aristoflex HMB from Clariant), Arylates/Beheneth-25 Methacrylate Copolymer (Aculyn 28 from Rohm and Haas); Acrylates/Steareth-20 Methacrylate Copolymer (Aculyn 22 from Rohm and Haas), PEG-150/Decyl Alclhol/SMDI Copolymer (Aculyn 44 from Rohm and Haas), PEG-150 Distearate (Aculyn 60 from Rohm and Haas), Acylates/Steareth-20 Methacrylate Crosspolymer (Aculyn 88 from Rohm and Haas), Aqupec SER-150 (acrylates/C10-30 alkyl acrylates crosspolymer) comprising about C18 (stearyl) side chains and about 0.4% hydrophobic modification (HM), Aqupec HV-701EDR which comprises about C8 (octyl) side chains and about 3.5% HM., Stabylen 30 (from 3V Sigma S.p.A), and Carbopol Aqua SF-1 (crosslinked acrylates copolymer) having average 4.5 carbon alkyl side chains and more than 50 HM.

The cleansing composition can also optionally include cationic polymers to improve the lathering and skin feel benefits of the cleansing composition during and after use. If present, the cleansing composition can include from about 0.001% to about 10%, alternatively from about 0.01% to about 5%, and alternatively from about 0.05% to about 1%, by weight of the composition, of a cationic polymer. Suitable cationic polymers for use in the antimicrobial bar composition include, but are not limited to, cationic polysaccharides; cationic copolymers of saccharides and synthetic cationic monomers; cationic polyalkylene imines; cationic ethoxy polyalkylene imines; cationic poly[N-[3-(dimethylammonio)propyl]-N′[3-(ethyleneoxyethylene dimethyl ammonio)propyl]urea dichloride]. Suitable cationic polymers generally include polymers having a quaternary ammonium or substituted ammonium ion.

The cleansing composition can further optionally include a hydrophilic polymer to improve skin feel benefits of the cleansing composition during and after use. For example, in one embodiment, the cleansing composition can include a nonionic polyethelye oxide polymer such as POLYOX™. If present, the cleansing composition can include from about 0.001% to about 10%, alternatively from about 0.01% to about 5%, and alternatively from about 0.05% to about 1%, by weight of the composition, of a hydrophilic polymer.

According to other example embodiments, the cleansing composition can optionally include from about 0.001% to about 10%, alternatively from about 0.01% to about 5%, and alternatively from about 0.05% to about 1%, by weight of the cleansing composition, of a deposition polymer. Suitable deposition polymers for use in the cleansing compositions disclosed herein can comprise cationic hydroxyethyl cellulosic polymers (polyquaternium 10, UCARE polymer JR400, LR400, JR30M, KG30M), cationic guar polymers (JAGUAR® from Rhodia (Jaguar C13S, Jaguar C14S, Jaguar C-17, Hi-Care 1000, Jaguar Excel, Jaguar CHT), and N-HANCE® polymers from Aqualon (N-Hance 3000, N-Hance 3196, N-Hance 3198, N-Hance 3205, N-Hance 3215, N-Hance 3269, N-Hance 3270), and synthetic acrylamide polymer (polyquaternium 76).

ii. Gums

The cleansing composition can also optionally include from about 0.01% to about 15%, alternatively from about 0.1% to about 10%, and alternatively 1% to about 5%, by weight of the cleansing composition, of a gum. Suitable gums for use in the cleansing composition disclosed herein can include carregeenan, xanthan, gellan, pectin, guar, alginate, wellan, pullulan and similar gums such as those outlined in “Industrial Gums Polysaccharides and Their Derivatives”, R. L. Whistler.

iii. Pluronics

In another example embodiment, the cleansing composition can include from about 5% to about 50%, alternatively from about 10% to about 40%, and alternatively 20% to about 30%, by weight of the cleansing composition of a pluronic. The pluronic can include, for example, an F87, F88, F98, F108, or an F127 pluronic.

iv. Salts

The cleansing composition can optionally include salts. In one embodiment, the salts can help bind the water in the cleansing composition thereby preventing water loss by evaporation or other means. Additionally, the salts can act as a preservative for other ingredients in the cleansing composition. The cleansing composition can optionally include from about 0.01% to about 15%, alternatively from about 1% to about 12%, and alternatively from about 2.5% to about 10.5%, by weight of the composition, of an inorganic salt. Suitable inorganic salts that can be included in the antimicrobial bar composition include magnesium nitrate, trimagnesium phosphate, calcium chloride, sodium carbonate, sodium aluminum sulfate, disodium phosphate, sodium polymetaphosphate, sodium magnesium succinate, sodium tripolyphosphate, aluminum sulfate, aluminum chloride, aluminum chlorohydrate, aluminum-zirconium trichlorohydrate, aluminum-zirconium trichlorohydrate glycine complex, zinc sulfate, zinc carbonate, ammonium chloride, ammonium phosphate, calcium acetate, calcium nitrate, calcium phosphate, calcium sulfate, ferric sulfate, magnesium chloride, magnesium sulfate, and the like.

v. Antimicrobial Agents

The cleansing composition can optionally further include one or more antibacterial agents that can serve to further enhance the antimicrobial effectiveness of the bar compositions. When present, the antimicrobial bar composition can include from about 0.001% to about 5%, alternatively from about 0.01% to about 2%, and alternatively from about 0.1% to about 1%, by weight of the cleansing composition, of the antimicrobial agent. Examples of antibacterial agents that can be used herein are the carbanilides, for example, triclocarban (also known as trichlorocarbanilide), triclosan, a halogenated diphenylether available as DP-300 from Ciba-Geigy, hexachlorophene, 3,4,5-tribromosalicylanilide, and salts of 2-pyridinethiol-1-oxide, to salicylic acid and other organic acids, and a pyrithione or a polyvalent metal salt of pyrithione such as a zinc salt of 1-hydroxy-2-pyridinethione (known as “zinc pyrithione” or “ZPT”) in platelet or fine particle form. Other suitable antibacterial agents are described in detail in U.S. Pat. No. 6,488,943 (referred to as antimicrobial actives).

vi. Brighteners

Additionally, brighteners can be included as optional ingredients in the antimicrobial bar composition at an amount of from about 0.001% to about 1%, preferably from about 0.005% to about 0.5%, and more preferably from about 0.01% to about 0.1%, by weight of the composition. Examples of suitable brighteners in the present compositions include disodium-4,4′-bis-(2-sulfostyril)-biphenyl (commercially available under the tradename Brightener-49, from Ciba Specialty Chemicals); disodium-4,4′-bis-[(4,6-di-anilino-s-triazine-2-yl)-amino]-2,2′-stilbenedisulfonate (commercially available under the tradename Brightener 36, from Ciba Specialty Chemicals); 4,4′-bis-[(4-anilino-6-morpholino-s-triazine-2-yl)-amino]-2,2′-stilbenedi-sulfonate (commercially available under the tradename Brightener 15, from Ciba Specialty Chemicals); and 4,4′-bis-[(4-anilino-6-bis-2(2-hydroxy-yethyl)-amino-s-triazine-2-yl)-amino]-2,2′-stilbenedisulfonate (commercially available under the tradename Brightener 3, from Ciba Specialty Chemicals); and mixtures thereof.

vii. Moisturizers and Benefit Agents

Moisturizers and/or benefit agents can also optionally be included in the cleansing composition to provide the skin conditioning benefits and to improve the mildness of the cleansing composition. The selection of the levels and types of moisturizers to be incorporated into the product is made without adversely affecting the stability of the product or its in-use characteristics, thereby delivering good moisturization and lather.

Examples of moisturizers suitable for use herein can include long chain fatty acids, liquid water-soluble polyols, glycerin, propylene glycol, sorbitol, polyethylene glycol, ethoxylated/propoxylated ethers of methyl glucose (e.g., methyl gluceth-20) and lanolin alcohol (e.g., Solulan-75).

Additionally, examples of benefit agents suitable for use herein can include petrolatum, lanolin, derivatives of lanolin (e.g. lanolin oil, isopropyl lanolate, acetylated lanolin, acetylated lanolin alcohols, lanolin alcohol linoleate, lanolin alcohol riconoleate) hydrocarbon oils (e.g. mineral oil) natural and synthetic waxes (e.g. micro-crystalline waxes, paraffins, ozokerite, lanolin wax, lanolin alcohols, lanolin fatty acids, polyethylene, polybutene, polydecene, pentahydrosqualene) volatile or non-volatile organosiloxanes and their derivatives (e.g. dimethicones, cyclomethicones, alkyl siloxanes, polymethylsiloxanes, to methylphenylpolysiloxanes), natural and synthetic triglycerides (e.g. castor oil, soybean oil, sunflower seed oil, maleated soy bean oil, safflower oil, cotton seed oil, corn oil, walnut oil, peanut oil, olive oil, cod liver oil, almond oil, avocado oil, palm oil, sesame oil) and combinations thereof. In one embodiment, at least about 50%, by weight of the benefit agent, can be selected from the group consisting of petrolatum, mineral oil, soy bean oil, paraffins, polyethylene, polybutene, polydecene, dimethicones, alkyl siloxanes, cyclomethicones, lanolin, lanolin oil, lanolin wax, sucrose polyester, and combinations thereof.

When moisturizers and/or benefit agents are used in the cleansing compositions of the cleansing composition disclosed herein, such moisturizers or benefit agents can be used at a concentration or level of from about 2% to about 20%, alternatively from about 4% to about 15%, and alternatively from about 8% to about 12% by weight of the cleansing composition.

viii. Electrolyte

According to one embodiment, the solid, dissolvable cleansing composition can also include one or more electrolytes. The solid, dissolvable cleansing composition can include from about 0.01% to about 5%, alternatively from about 0.05% to about 2%, and alternatively about 0.1% to about 1%, by weight of the dissolvable, cleansing composition, of an electrolyte. In example embodiments, the electrolyte useful in the solid, dissolvable cleansing composition disclosed herein can include an anion comprising phosphate, chloride, sulfate or citrate and a cation comprising sodium, ammonium, potassium, magnesium or mixtures thereof.

Other optional ingredients in the cleansing composition can include: perfumes or fragrances; sequestering agents; preservatives such as citric acid and Kathon; coloring agents, or mixtures thereof typically in an amount of 0.01 to 1%, preferably 0.01 to 0.05%, by weight of the composition. The other optional ingredients in the cleansing composition can also include thickening agents; chelators (e.g. such as those described in U.S. Pat. No. 5,487,884 issued to Bisset, et al.); sequestrants; vitamins (e.g. Retinol); vitamin derivatives (e.g. tocophenyl actetate, niacinamide, panthenol); sunscreens; desquamation actives (e.g. such as those described in U.S. Pat. Nos. 5,681,852 and 5,652,228 issued to Bisset); anti-wrinkle/anti-atrophy actives (e.g. N-acetyl derivatives, thiols, hydroxyl acids, phenol); anti-oxidants (e.g. ascorbic acid derivatives, tocophenol) skin soothing agents/skin healing agents (e.g. panthenoic acid derivatives, aloe vera, allantoin); skin lightening agents (e.g. kojic acid, arbutin, ascorbic acid derivatives) skin tanning agents (e.g. dihydroxyacteone); anti-acne medicaments; essential oils; sensates; pigments; colorants; pearlescent agents; interference pigments (e.g such as those disclosed in U.S. Pat. No. 6,395,691 issued to Liang Sheng Tsaur, U.S. Pat. No. 6,645,511 issued to Aronson, et al., U.S. Pat. No. 6,759,376 issued to Zhang, et al, U.S. Pat. No. 6,780,826 issued to Zhang, et al.) particles (e.g. talc, kolin, mica, smectite clay, cellulose powder, polysiloxane, silicas, carbonates, titanium dioxide, polyethylene beads) hydrophobically modified non-platelet particles (e.g. hydrophobically modified titanium dioxide and other materials described in a commonly owned, patent application published on Aug. 17, 2006 under Publication No. 2006/0182699A, entitled “Personal Care Compositions Containing Hydrophobically Modified Non-platelet particle filed on Feb. 15, 2005 by Taylor, et al.), and mixtures thereof.

### Tests/Methods

As described above, the cleansing composition disclosed herein can include and/or exhibit specific physical properties as defined by the Hand Dissolution Test and Oscillatory Rheology Test described below.

Hand Dissolution Test

The solid, dissolvable cleansing composition disclosed herein includes a dissolution rate that allows the cleansing composition and, in particular, the expandable microspheres that can be included therein to disintegrate during use with water. The dissolution rate of the cleansing composition can be determined according to the following:

Place approximately 0.5 g of the cleansing composition in the palm of the hand while wearing nitrile gloves. Quickly apply 7.5 cm³ of luke warm tap water (from about 30° C. to about 35° C.) to the cleansing composition via a syringe. Using a circular motion, rub the palms of the hands together 2 strokes at a time until dissolution occurs (up to 30 strokes). Then, determine the hand dissolution value as the number of strokes it takes for complete dissolution or as 30 strokes as the maximum (in the case for where the solid is considered non-dissolving).

The solid, dissolvable cleansing composition disclosed herein can have a hand dissolution value of from about 1 to about 30 strokes, alternatively from about 2 to about 25 strokes, alternatively from about 3 to about 20 strokes, and alternatively from about 4 to about 15 strokes.

Rheology Test

In one embodiment, to measure the viscosity of the cleansing composition, use a AR G2 Rheometer (TA Instruments, DE, USA) equipped with cross hatched lower and upper geometry, where the diameter of the upper geometry is 40 mm Place approximately 0.25 grams of cleansing composition onto the lower test geometry and manually applying force to create an even flat surface. Then, lower the upper geometry down onto the cleansing composition until the upper geometry is in full contact with the cleansing composition. After lowering the upper geometry down, conduct a steady state shear stress test over a stress range of 1.0 to 10,000 Pa at to a temperature of 25° C. to determine the viscosity.

### Lather Volume Test

Lather volume of a personal care composition, is measured using a graduated cylinder and a rotating apparatus. A 1,000 ml graduated cylinder is used which is marked in 10 ml increments and has a height of 14.5 inches at the 1,000 ml mark from the inside of its base (for example, Pyrex No. 2982). Distilled water (100 grams at 25° C.) is added to the graduated cylinder. The cylinder is clamped in a rotating device, which clamps the cylinder with an axis of rotation that transects the center of the graduated cylinder. Weight 0.1 grams of the composition and place into the graduated cylinder onto the side of the cylinder, above the water line, and cap the cylinder. The cylinder is rotated for 20 complete revolutions at a rate of about 10 revolutions per 18 seconds, and stopped in a vertical position to complete the first rotation sequence. A timer is set to allow 15 seconds for lather generated to drain. After 15 seconds of such drainage, the first lather volume is measured to the nearest 10 ml mark by recording the lather height in ml up from the base (including any water that has drained to the bottom on top of which the lather is floating).

If the top surface of the lather is uneven, the lowest height at which it is possible to see halfway across the graduated cylinder is the first lather volume (ml). If the lather is so coarse that a single or only a few foam cells which comprise the lather (“bubbles”) reach across the entire cylinder, the height at which at least 10 foam cells are required to fill the space is the first lather volume, also in ml up from the base. Foam cells larger than one inch in any dimension, no matter where they occur, are designated as unfilled air instead of lather. Foam that collects on the top of the graduated cylinder but does not drain is also incorporated in the measurement if the foam on the top is in its own continuous layer, by adding the ml of foam collected there using a ruler to measure thickness of the layer, to the ml of foam measured up from the base. The maximum lather height is 1,000 ml (even if the total lather height exceeds the 1,000 ml mark on the graduated cylinder). 30 seconds after the first rotation is completed, a second rotation sequence is commenced which is identical in speed and duration to the first rotation sequence. The second lather volume is recorded in the same manner as the first, after the same 15 seconds of drainage time. A third sequence is completed and the third lather volume is measured in the same manner, with the same pause between each for drainage and taking the measurement.

The lather results after each sequence are added together and the Total Lather Volume determined as the sum of the three measurements, in milliters (“ml”). The Flash Lather Volume is the result after the first rotation sequence only, in ml, i.e., the first lather volume. Compositions according to the present invention perform significantly better in this test than to similar compositions in conventional emulsion form.

### Methods of Manufacture

The cleansing composition of the present invention can manufactured by mixing the expandable microspheres into a liquid cleansing and/or benefit phase including, for example, surfactants, benefit agents, preservatives, dyes and other soluble components. In example embodiments, the phases can be mixed using standard manufacturing equipment including, but not limited to, planetary mixers, ribbon blenders, extruders, and or a roll mill. The cleansing composition can then be packaged as a bulk paste or cream or alternatively cut and packaged as individual unit doses. The cleansing composition can also be optionally dried, molded, and/or calendared to provide a specific shape.

### Methods of Use

The present invention also relates to a method of cleansing the skin or hair with a dissolvable, personal cleansing composition of the present invention. Such methods include: wetting with water a dissolvable, personal cleansing composition disclosed herein; and applying the wetted personal cleansing composition to a surface such as skin or hair.

As described above, the dissolvable, personal cleansing composition disclosed herein are intended to be wetted with water prior to use. According to example embodiments, dissolvable personal cleansing composition can be wetted by immersion in water or by placing it under a stream of water. When the articles of the present invention comprise a surfactant such as the surfactants disclosed herein, a liquid form of the personal cleansing composition can be created that generates lather and cleansing characteristics. During the cleansing process and subsequent rinsing with water, any therapeutic or aesthetic benefit agents that can be included in the dissolvable, cleansing composition can be deposited onto the skin or hair.

### EXAMPLES

The following examples further describe and demonstrate embodiments within the scope of the present invention. In the following examples, all ingredients are listed at an active level. The examples are given solely for the purpose of illustration and are not to be construed as limitations of the personal cleansing article or components thereof such as the viscoelastic cleansing composition or water insoluble substrate or wrap, as many variations thereof are possible without departing from the spirit and scope disclosed herein.

### Example 1

A solid, dissolvable personal cleansing composition is prepared by making a to homogenous mixture of each of the ingredients listed in Example 1, except the Expancel. Then, add the Expancel and speed mix using, for example, a speed mixer such as a Hauschild Engineering model DAV 400 FV, Germany at 2000 rpm for 30 seconds. Hand mix from top to bottom and then, speed mix for an additional 30 seconds at 2000 rpm. The calculated active levels of surfactants and Expancel are included in parenthesis.

| Ex. 1 | As added % | (active %) |
|---|---|---|
| Sodium Laureth-1 Sulfate (70% active) | 45.71 | (32) |
| Cocoamidopropyl Betaine (30% active) | 11.67 | (3.5) |
| EDTA | 1.0 | |
| Preservatives | 1.3 | |
| Colorant | 0.1 | |
| Expancel 091 We 40 D24 (15% active) | 40.0 | (6) |

| | Ex. 2 | Ex. 3 | Ex. 4 | Ex. 5 |
|---|---|---|---|---|
| Sodium Tridecyl Ether-2 Sulfate | 28.0 | | | |
| (65% active) | (18.2) | | | |
| Sodium Cocoyl Isethionate | | 32.0 | 28.0 | 25.0 |
| (65% active) | | (20.8%) | (18.2) | (16.25) |
| Cocoamidylpropyl Betaine | 10.0 | | 10.0 | 2.0 |
| (30% active) | (3) | | (3) | (0.6) |
| Sodium Lauroyl Sarcosinate | | 10 | | 6.0 |
| (30% active) | | (3) | | (1.8) |
| Glycerin | 7.0 | 3.0 | 3.0 | 3.6 |
| Preservatives | 0.7 | 0.8 | 1.1 | 0.8 |
| EDTA | 0.1 | 0.1 | 0.1 | 0.1 |
| Expancel 091 WE 40 D24 | 38.0 | 41.0 | 43.0 | 45.0 |
| (15% active) | (5.7) | (6.15) | (6.45) | (6.75) |
| N-Hance 3215 | 0.2 | 0.6 | 0.8 | 0.5 |
| (from Aqualon) | | | | |
| Petrolatum | 8.0 | 5.0 | 6.0 | 8.0 |
| (G2218 from Sonnoborne) | | | | |
| Sefose 1618S | 8.0 | 7.5 | 8.0 | 9.0 |
| (from P&G Chemical) | | | | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cleansing composition comprising:
about 15% or more, by weight of the cleansing composition, of a surfactant;
about 0.5% or more, by weight of the cleansing composition, of a co-surfactant; and
about 5% or more, by weight of the cleansing composition, of expandable microspheres,
wherein the cleansing composition comprises a viscosity of about 1,000,000 to about 50,000,000 Pascal-seconds (PaS).

2. The cleansing composition of claim 1, wherein the composition is a dissolvable solid.

3. The cleansing composition of claim 1, wherein the composition comprises about 20% or more, by weight of the cleansing composition, of a surfactant.

4. The cleansing composition of claim 1, wherein the surfactant comprises at least one of SLEnS where n is about 1 to about 3 and STnS where n is about 1 to about 3.

5. The cleansing composition of claim 1, wherein the co-surfactant comprises a betaine.

6. The cleansing composition of claim 1, wherein the expandable microspheres comprise hollow deformable particles.

7. The cleansing composition of claim 6, wherein the expandable microsphere comprises an expanded copolymer of vinylidene chloride and acrylonitrile; or vinylidene chloride, acrylonitrile, and methacrylate; or a non-expanded copolymer of vinylidene chloride and acrylonitrile; or vinylidene chloride, acrylonitrile and methacrylate.

8. The cleansing composition of claim 1, wherein the cleansing composition further comprises from about 0.01% to about 5%, by weight of the cleansing composition, of an electrolyte.

9. The cleansing composition of claim 1, wherein the cleansing composition further comprises a benefit agent.

10. A solid, dissolvable cleansing composition comprising:
about 25% or more, by weight of the cleansing composition, of a surfactant;
about 3% or more, by weight of the cleansing composition, of a co-surfactant; and
about 5% or more, by weight of the cleansing composition, of expandable microspheres,
wherein the expandable microspheres comprise hollow deformable particles of an expanded copolymer of vinylidene chloride and acrylonitrile; or of vinylidene chloride, acrylonitrile and methacrylate; or a non-expanded copolymer of vinylidene chloride and acrylonitrile; or of vinylidene chloride, acrylonitrile and methacrylate; and
wherein the cleansing composition comprises a viscosity of about 1,000,000 to about 10,000,000 Pascal-seconds (PaS).

11. The cleansing composition of claim 10, wherein the surfactant comprises at least one of the following: sodium laureth-n sulfate (SLEnS) where n is about 1 to about 3 and sodium trideceth-n sulfate (STnS) where n is about 1 to about 3.

12. The cleansing composition of claim 11, wherein the co-surfactant comprises a betaine.

13. The cleansing composition of claim 12, wherein the expandable microspheres comprise an active content of about 8% or more.

14. The cleansing composition of claim 13, wherein the cleansing composition further comprises from about 0.01% to about 5%, by weight of the cleansing composition, of an electrolyte.

15. The cleansing composition of claim 14, wherein the cleansing composition further comprises a benefit agent.

16. The cleansing composition of claim 15, wherein the cleansing composition further comprises a polymer.

17. The cleansing composition of claim 16, wherein the polymer comprises at least one of the following: a hydrophobic ally modified polymer, a cationic polymer, a hydrophilic polymer, and a deposition polymer.

18. A solid, dissolvable cleansing composition comprising:
about 30% or more, by weight of the cleansing composition, of a surfactant, and wherein the surfactant comprises sodium laureth-n sulfate (SLEnS) where n is about 1 to about 3;
about 5% or more, by weight of the cleansing composition, of a co-surfactant, wherein the co-surfactant comprises a betaine; and
about 6% or more, by weight of the cleansing composition, of expandable microspheres, wherein the expandable microspheres comprise hollow deformable particles of an expanded copolymer of vinylidene chloride and acrylonitrile or of vinylidene chloride, acrylonitrile and methacrylate or a non-expanded copolymer of vinylidene chloride and acrylonitrile or of vinylidene chloride, acrylonitrile and methacrylate, and
wherein the cleansing composition comprises a viscosity of about 1,000,000 to about 10,000,000 Pascal-seconds (PaS).
